(19) United States
(12) Patent Application Publication
(10) Pub. No.: US 2006/0222715 A1
(43) Pub. Date: Oct. 5, 2006

(54) Particles containing an acitve agent in the form of a co-precipitate

(76) Inventors: Gerard Cousin, Villemeux Sur Eure (FR); Gael Lamoureux, Le Boullay Thierry (FR)

Correspondence Address: FOLEY AND LARDNER LLP;SUITE 500, 3000 K STREET NW, WASHINGTON, DC, 20007, US

(21) Appl. No.: 10/564,786

(22) PCT Filed: Jul. 16, 2004
(86) PCT No.: PCT/FR04/01878

§ 371 Date: Jun. 5, 2006

(30) Foreign Application Priority Data
Jul. 17, 2003 (FR) 03/08720

(51) Int. Cl. A61K 9/14

(52) U.S. Cl. 424/489

(57) ABSTRACT

The invention relates to particles containing an active agent in the form of a co-precipitate, to a method for producing said particles and to pharmaceutical forms containing said particles.

The invention relates to particles comprising an active substance in the form of a coprecipitate, to the process for the preparation of said particles and to the pharmaceutical forms comprising these particles.

The preparation of oral medicaments is restricted with increasingly complex synthetic molecules often exhibiting physicochemical characteristics unfavorable to the absorption of the molecule in the gastrointestinal tract and in particular a very low solubility in aqueous media.

This is often reflected by a very low bioavailability of these molecules and the need to administer high doses of active substance to the patients in order to achieve the effective concentration.

This is all the more detrimental when the effective concentration to be achieved is high, when the number of tablets or gelatin capsules to be taken at one time is high or when the number of times per day they have to be taken is more frequent.

The preparation of solid dispersions is one of the methods which make it possible to substantially improve the solubility of the active principles and consequently their bioavailability.

The solid dispersions are generally obtained according to two methods, on the one hand by evaporating a solution comprising the active substance and the inert carrier and, on the other hand, by melting the two above compounds and then solidifying.

In the first case, the product is a coprecipitate; in the second case, it is a comelt.

A first difficulty is that of obtaining particles having the fastest possible rate of dissolution and the highest possible dissolved fraction, in order to ensure satisfactory bioavailability of the molecule.

A second difficulty is that of obtaining particles having a narrow particle size distribution and exhibiting a size compatible with putting into a form intended for oral administration, while retaining a high content of active substance.

A third difficulty is that of obtaining a formulation which is stable over time under unfavorable temperature and humidity conditions, such as those used in stability studies, for example 30° C./60% RH (relative humidity) or 40° C./75% RH.

The solid dispersions are generally composed of an active substance dispersed in the molecular state in a hydrophilic agent, for example a polymer.

International application WO 97/04749 has as subject matter a process for the preparation of solid dispersions in which the active substance(s) is (are) dissolved in an organic solvent additionally comprising a very hydrophilic cyclic amide and advantageously a surface-active agent, the organic solution thus obtained subsequently being evaporated to dryness, followed by milling and sieving. The cyclic amide is a polyvinylpyrrolidone having a molecular weight varying from 10 000 to 50 000.

Application EP 1 103 252 discloses pharmaceutical compositions comprising particles having a core covered with a single layer including an antifungal active principle, such as itraconazole, said layer being obtained by spraying a solution comprising the active principle, a hydrophilic polymer and a nonionic surfactant. These compositions, obtained by conventional techniques, improve the solubility of the active principle.

In extreme cases, the preparation of a solid dispersion is not in itself sufficient to significantly improve the bioavailability.

The Applicant Company has shown that it is entirely possible to substantially improve the bioavailability of active substances which are virtually insoluble in water and to increase the rate of dissolution thereof and the fraction thereof dissolved in vitro or in vivo by preparing a particle composed of a coprecipitate applied as a layer around a neutral hydrophilic carrier and comprising at least one active substance, one surface-active agent and one hydrophilic polymer.

The layer formed around the carrier is a solid dispersion in the form of a coprecipitate which is readily soluble in water or physiological media, for example the gastric fluid.

Furthermore, the Applicant Company has shown that it is entirely possible to substantially improve the problems of agglomeration of particles encountered in the cases of spraying large amounts of coating solutions by milling the particles not only at the end of the spraying stage but during this stage.

The reduction in size resulting from the milling then makes it possible to continue the spraying over smaller particles less liable to agglomerate with one another than particles exhibiting uniform growth during a single spraying stage.

The coprecipitate is obtained by spraying an organic solution over a neutral hydrophilic carrier, said solution comprising at least one active substance, one surface-active agent and one hydrophilic polymer, said spraying being characterized in that the spraying of the whole of the solution is carried out in at least two separate stages, each of these stages being followed systematically by a stage of milling the product obtained on conclusion of the preceding stage.

The process for the preparation of the particles advantageously comprises the following stages:
a) preparing an organic solution comprising the active substance, the hydrophilic polymer and the surface-active agent,
b) spraying a portion of the solution obtained in a) over the neutral hydrophilic carriers,
c) milling the particles obtained in stage b),
d) spraying the remaining amount of the organic solution over the particles milled in stage c), and
e) final milling of the particles obtained in stage d).

The spraying/milling sequence (stages b to d) can be repeated one or more times according to the volume of solution to be sprayed and the kinetics of growth of the particles during the spraying stage.

The whole of the organic solution can be prepared all at once; however, in order to avoid excessively great evaporation of the organic solvents, it is preferable to prepare each fraction of the solution immediately before the spraying stage.

The spraying of the organic solution can be carried out in a coating pan, in a perforated pan coater or in a fluidized bed.

In a preferred embodiment of the process in accordance with the invention, all the spraying stages are carried out in a fluidized bed equipped with an explosion-suppressing device.

The fluidized bed is equipped with a spray nozzle, the position and the orientation of which for spraying can be chosen.

This choice makes it possible to control the kinetics of growth of the particles and to prevent sticking phenomena related to the nature of the active substance, to the qualitative and quantitative composition of the coating solution sprayed and to the various processing parameters (for example, the temperature, the air pressure and the spraying throughput).

The particles are usually dried after the spraying of the organic solution.

The drying can be carried out on trays or directly in the equipment used for the spraying stage.

The drying can be carried out either immediately after the spraying of the organic solution and before the milling or immediately after the milling of the particles.

The milling can be carried out on any type of device intended for this purpose, which can be a mill of oscillating type or a mill equipped with pins.

The rotary mill of Fitzmill type or the oscillating mill of Frewitt type is equipped with a rotor which forces the particles through a screen with graded openings.

The mill of Forplex type is equipped with pins against which the particles are hurled at high speed.

The mill used between each spraying stage can be different from that used for the final milling.

The process of the invention is particularly suitable for active substances having very little solubility in water, that is to say active substances which are partially soluble in 1000 parts of water or more than 1000 parts of water, preferably for active substances which are virtually insoluble in water, that is to say those which are partially soluble in 10 000 parts of water or more than 10 000 parts of water.

The active substance or substances can be chosen from any family of compounds, for example from gastrointestinal sedatives, antacids, analgesics, anti-inflammatories, coronary vasodilators, peripheral and cerebral vasodilators, anti-infectives, antibiotics, antivirals, antiparasitics, anticancers, anxiolytics, neuroleptics, stimulants of the central nervous system, antidepressants, antihistaminics, antidiarrheals, laxatives, nutritional supplements, immunodepressants, hypocholesterolemics, hormones, enzymes, antispasmodics, antianginals, medicaments which influence the heart rate, medicaments used in the treatment of arterial hypertension, antimigraines, medicaments which influence blood clotting, antiepileptics, muscle relaxants, medicaments used in the treatment of diabetes, medicaments used in the treatment of thyroid dysfunctions, diuretics, anorectics, antiasthmatics, expectorants, antitussives, mucoregulators, decongestants, hypnotics, antinauseants, hematopoietics, uricosurics, plant extracts, contrast agents or any other family of compounds, it being possible for the active substances used in combination in the tablet to be chosen from the same family or from different families.

The active substances can be provided in the form of their pharmaceutically acceptable salts or any polymorphic form (racemic, enantiomeric, and the like). The term “pharmaceutically acceptable salts” is understood to mean the derivatives of the compounds described in which the pharmaceutically active base compound is converted to its salt with a base or acid, examples of pharmaceutically active salts comprising in particular organic or inorganic acid salts of basic residues, such as amines, or alkali metal derivatives or organic salts of acidic residues, such as carboxylic acids, and the like.

The active substance is present in the particle in a proportion which can vary between 1 and 60% by weight.

The inert hydrophilic carrier can be composed of any chemically and pharmaceutically inert excipient existing in the crystalline or amorphous particulate form, for example derivatives of sugars, such as lactose, preferably extra fine lactose (EFK), sucrose or hydrolyzed starch (maltodextrins); celluloses or mixtures, such as sucrose and starch, or cellulose-based mixtures can also be used for the preparation of inert spherical carriers.

The inert hydrophilic carrier is present in a proportion which can range up to 95% by weight.

The unit particle size of the inert hydrophilic carrier can be between 50 and 500 μm, preferably between 90 and 200 μm.

The hydrophilic polymer can be chosen from polyvinyl-pyrrolidones and cellulose derivatives, acrylic polymers and polyethylene glycols.

The polyvinylpyrrolidone can be chosen from polymers with a molecular weight of between 10 000 and 50 000.

The cellulose derivative is chosen from hydroxylated derivatives, for example hydroxypropylmethylcellulose, hydroxypropylcellulose, hydroxymethylcellulose, hydroxypropylmethylcellulose phthalate and hydroxy-propylmethylcellulose acetate/succinate.

The preferred hydroxypropylmethylcellulose is chosen from those having an apparent viscosity of between 2.4 and 18 cP and more preferably still of between 2.4 and 5 cP.

The acrylic polymer can be chosen from the ammoniomethacrylate copolymer, polyacrylate, poly-methacrylate and the methacrylic acid copolymer.

The polyethylene glycol can be chosen from polymers with a molecular weight of between 1000 and 20 000.

The hydrophilic polymer/active principle ratio by weight is preferably between 10/1 and 1/2.

The surface-active agent can be chosen from cationic, anionic, nonionic or amphoteric agents, alone or as a mixture.

The surface-active agent can be chosen, for example, from compounds such as sodium lauryl sulfate, the monooleate, the monolaurate, the monopalmitate, the monostearate, the trioleate, the tristearate or any other ester of polyoxyethylenated sorbitan, preferably Tween® 20, 40, 60 or 80, glycerides of polyoxyethylenated fatty acids, these fatty acids being saturated or unsaturated and composed of at least 8 carbon atoms, poloxamers, such as poloxamer 188, ethylene oxide/propylene oxide block copolymers, such as Pluronic® F68 or F87, lecithin, stearyl alcohol, cetearyl alcohol, cholesterol, polyoxyethylenated castor oil, fatty alcohol polyoxyethylenated ethers, such as the Brij® products, and polyoxyethylenated stearates.

The surface-active agent is advantageously present in a proportion which can vary between 0.1 and 20% by weight, with respect to the total weight obtained.

The organic solvent can be chosen from ethanol, isopropanol, tetrahydrofuran, isopropyl ether, acetone, methyl ethyl ketone, methylene chloride or a mixture of these solvents.

The volume of solvent takes into account the solubility of the various components of the organic solution.

Another subject matter of the present invention is a particle composed of a coprecipitate which is applied as a layer around a carrier and which comprises at least one active substance, one surface-active agent and one hydrophilic polymer, capable of being obtained by spraying a solution comprising at least one active substance, one surface-active agent and one hydrophilic polymer, said spraying being carried out at least in two separate stages, said stages each being followed by a milling stage.

It results from the process of the invention that, if the carrier over which the solution comprising at least one active substance, one surface-active agent and one hydrophilic polymer is sprayed is, in the first stage, entirely devoid of any active substance, the carrier resulting from the first milling over which the same solution is sprayed is composed both of the neutral hydrophilic carrier and of the coprecipitate comprising at least one active substance, one surface-active agent and one hydrophilic polymer.

The content of active principle of the carrier used for applying the solution comprising at least one active substance, one surface-active agent and one hydrophilic polymer thus increases after each spraying/milling cycle.

After the final spraying stage, the structure of the particles is a sphere composed of a core comprising a mixture of neutral hydrophilic carrier and of coprecipitate comprising at least one active substance, one surface-active agent and one hydrophilic polymer and of an external layer of the same mixture comprising at least one active substance, one surface-active agent and one hydrophilic polymer.

The size of the particles is usually between 50 and 1000 μm, preferably between 100 and 800 μm, more preferably still between 200 and 500 μm, and is determined by conventional methods, for example using a set of sieves with graded mesh openings or by diffraction of a laser.

In a preferred implementation of the invention, the particles of the invention comprise:
from 15 to 40% by weight of an inert hydrophilic carrier, preferably EFK lactose,
from 15 to 30% by weight of active substance,
from 30 to 60% by weight of hydrophilic polymer, preferably HPMC,
from 1 to 10% by weight of surface-active agent, preferably a nonionic agent chosen from the group consisting of polysorbates 20 to 80.

The particles according to the invention can be used directly or mixed with excipients used in the pharmaceutical field for the preparation of a pharmaceutical form intended to be administered orally, such as, for example, a gelatin capsule or a tablet, and compatible chemically with the active substance or substances.

These excipients can be chosen from diluents, disintegrating agents, swelling agents, lubricants, antistatic agents, binders or adjuvants.

The diluent can be chosen from sugars, such as sucrose, lactose, fructose, dextrose or polyols of less than 13 carbon atoms, such as mannitol, xylitol, sorbitol, maltitol, lactitol or erythritol, dicalcium phosphate, tricalcium phosphate or a microcrystalline cellulose, alone or as a mixture.

The diluent is used in a proportion of between 20 and 90% by weight, preferably between 30 and 60% by weight, calculated with respect to the weight of the tablet.

The diluent is preferably used in its directly tabletable form, the mean diameter of the particles of which is from 100 to 500 μm, or in the form of a powder, the mean diameter of the particles of which is less than 100 μm, said powder being used alone or as a mixture with the directly tabletable product.

The disintegrating agent is chosen from the group consisting in particular of crosslinked sodium carboxymethylcellulose, denoted by the term croscarmellose, crosslinked polyvinylpyrrolidones, denoted by the term crospovidone, and their mixtures.

The disintegrating agent is used in a proportion of between 1 and 20% by weight, preferably between 5 and 15% by weight, calculated with respect to the weight of the tablet.

The swelling agent is chosen from the group consisting of microcrystalline cellulose, starches, modified starches, such as sodium starch glycolate or carboxymethyl starch, alginic acid or sodium alginate, and their mixtures.

The swelling agent is used in a proportion of between 1 and 15% by weight, calculated with respect to the weight of the tablet.

The lubricant is chosen from the group consisting of magnesium stearate, stearic acid, sodium stearyl fumarate, polyethylene glycols, sodium benzoate, a pharmaceutically acceptable oil, preferably dimethicone or liquid paraffin, and their mixtures.

The lubricant is used in a proportion which can range up to 2% by weight, preferably between 0.02 and 2% by weight, more preferably still between 0.5 and 1% by weight, calculated with respect to the weight of the tablet.

According to a first alternative form, the lubricant is incorporated in its entirety in the mixture of tableting excipients; in a second alternative form, a fraction of this lubricant is sprayed over the walls of the die and the punches during tableting, the lubricant then being in the form of a powder, for example magnesium stearate, or of a liquid, for example liquid paraffin.

The amounts of lubricant used in the internal and/or external phase are adjusted with care so as to prevent an excess from detrimentally affecting the cohesion of the powder bed during the tableting.

The antistatic agent can be chosen from the group consisting of micronized talc, unmicronized talc, colloidal silica (Aerosil®), treated silica (Aerosil® R972), precipitated silica (Syloid® FP244) and their mixtures.

The antistatic agent is used in a proportion which can range up to 5% by weight, calculated with respect to the weight of the tablet.

The binder is used in the dry form and can be a starch, a sugar, polyvinylpyrrolidone or carboxymethyl-cellulose, alone or as a mixture.

The binder is used in a proportion which can range up to 15% by weight, preferably of less than 10% by weight, calculated with respect to the weight of the tablet.

Adjuvants can also be added to the mixture intended to be put into gelatin capsules or to be tableted and are chosen from the group consisting of pH-adjusting agents, systems which make it possible to produce effervescence, in particular generators of carbon dioxide of the type of those used as pH-adjusting agents, or surfactants.

Another subject matter of the invention is pharmaceutical forms comprising the particles in accordance with the invention.

The preparation of the pharmaceutical form comprising the particles of the invention can comprise the following stages:
dry mixing the particles, obtained according to the process described above, with excipients,
tableting the mixture or placing the mixture in gelatin capsules in order to obtain a unit form.

In an advantageous embodiment, all the lubricant is sprayed over the punches and/or over the internal face of the tableting dies; the second stage of the mixing is then, of course, dispensed with.

In another advantageous method of preparation of the pharmaceutical form, the preparation of the mixture comprises two stages, the first stage, which consists in mixing the active substance with all the tableting excipients, except the internal lubricant, and then a second stage, in which the lubricant is added to the first mixture in its entirety or in part, the remaining part then being sprayed over the punches and/or over the internal face of the tableting dies.

The tableting of the mixture can be carried out on an alternating or rotary tablet press.

The hardness of the tablet is adjusted in order to make it possible to obtain a friability, measured according to the method of the European Pharmacopoeia, of less than 2%, preferably 1%.

The tablets can be round, oval or oblong in shape, can exhibit a flat, concave or convex surface and can optionally exhibit engravings or be beveled.

The tablets generally have a weight of between 0.1 gram and 2.0 grams and a size with a diameter of between 6 mm and 18 mm.

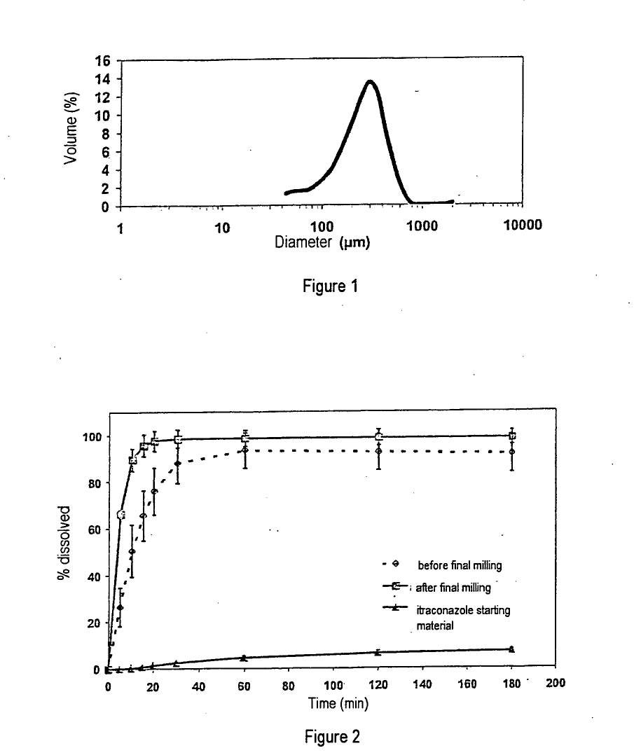

The example and FIGS. 1 to 4 which follow illustrate the invention.

FIG. 1 illustrates the particle size distribution, studied by laser particle sizing, of the particles prepared according to the process of the invention.

FIG. 2 represents the kinetics of dissolution of itraconazole alone (▴) and in the form of particles prepared according to the process of the invention, before final milling (⋄) and after final milling (▪).

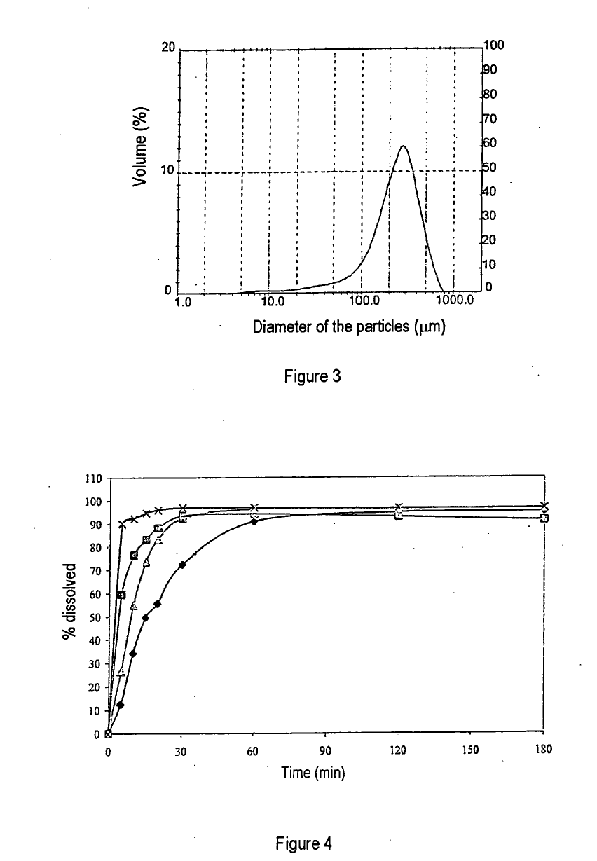

FIG. 3 illustrates the particle size distribution, studied by laser particle sizing, of the surfactant-free particles.

FIG. 4 illustrates the kinetics of dissolution of itraconazole before milling, with (▪) and without (♦) surfactant, and after milling, with (×) and without (▴) surfactant.

### EXAMPLE 1

1. Preparation of the Particles Comprising a Surfactant

Manufacturing is carried out on the GPCG1 fluidized bed in bottom spray mode.

The spraying solution is prepared by dissolving itraconazole (supplied by Wickoff) in a mixture of solvents, 96° alcohol/methylene chloride in a 41.6/58.4 ratio by weight, with HPMC 2910 5 cPs (supplied by Dow Chemical) and polysorbate 20 (Montanox® 20, supplied by Seppic).

The EFK lactose (supplied by HMS) is introduced into the fluidized bed and the solution is sprayed in bottom spray mode; 4 stages of spraying the solution are carried out successively; the granules are dried in the fluidized bed and milled using a Forplex mill with a 630 μm screen for the present use after each spraying stage.

After the fourth stage, the granules obtained are dried in the fluidized bed.

The coating parameters are presented in table 1:

TABLE 1

| | Coatings | | | | | | | |
|---|---|---|---|---|---|---|---|---|
| | 1st stage | | 2nd stage | | 3rd stage | | 4th stage | |
| Spraying | 45 | g/min | 39 | g/min | 39 | g/min | 39 | g/min |
| throughput | | | | | | | | |
| Spraying pressure | 0.15 | MPa | 0.15 | MPa | 0.15 | MPa | 0.15 | MPa |
| Amount of solution | 2.7 | kg | 3.1 | kg | 2.7 | kg | 2.5 | kg |
| sprayed | | | | | | | | |
| Set inlet air | 50-60° | C. | 48-56° | C. | 56-58° | C. | 52-54° | C. |
| Inlet air | 42-50° | C. | 50-56° | C. | 55-59° | C. | 51-55° | C. |
| temperature | | | | | | | | |
| Temperature of the | 29-33° | C. | 27-34° | C. | 27-34° | C. | 28-33° | C. |
| product | | | | | | | | |

The total weight of solution sprayed is 11 kg and the total granulating time is 4 h 47 min.

The particles obtained are dried in a GPCG1 fluidized bed, the drying parameters being as follows:
inlet air temperature: 48° C.,
drying time: 30 min,
cooling until the temperature reaches 29° C.

The particles are subsequently milled using a mill of Forplex type equipped with a screen with an opening of 630 μm.

The amount of dry granules obtained is 1.406 kg, corresponding to a yield of 93%.

The manufacturing formulation tested is shown in table 2.

TABLE 2

| Starting materials | Amount (g) | | % |
|---|---|---|---|
| EFK lactose | 600 | | 39.7 |
| Itraconazole | 350 | | 23.2 |
| HMPC 2910 5 cPs | 525 | | 34.8 |
| Polysorbate 20 | 35 | | 2.3 |
| 96° Alcohol | 4200 | | / |
| Methylene chloride | 5900 | | / |
| Amount of solution sprayed | 11 010 | | / |
| Theoretical dry weight | 1510 | | / |
| Theoretical assay | 231.79 | mg/g | / |

2. Results

2.1 Particle Size Distribution

It is presented in FIG. 1. The particle size distribution gives the following results:
D_{10%}: 62 μm
D_{50%}: 231 μm
D_{90%}: 419 μm.

2.2 Kinetics of Dissolution

They are studied with regard to three particulate bodies.

The measurements are carried out continuously under UV radiation, at a measuring wavelength of 254 nm, on 50 mg of itraconazole in 900 ml of D type 1.2 medium (paddle speed 100 rpm).

The results are illustrated in FIG. 2.

The coating produced according to the process of the invention considerably improves the dissolution of the itraconazole. The milling accelerates the dissolution: 50% of the product is dissolved after testing for 10 minutes for the unmilled fraction and after 4 min for the milled fraction.

### EXAMPLE 2

1. Preparation of Particles Not Comprising Surfactant

The particles are prepared according to the procedure described in example 1. The manufacturing formulation is shown in table 3.

TABLE 3

| Starting materials | Amount (g) | | % |
|---|---|---|---|
| EFK lactose | 600 | | 40.68 |
| Itraconazole | 350 | | 23.73 |
| HMPC 2910 5 cPs | 525 | | 35.59 |
| Polysorbate 20 | 0 | | 0 |
| 96° Alcohol | 4200 | | / |
| Methylene chloride | 5900 | | / |
| Amount of solution sprayed | 10 975 | | / |
| Theoretical dry weight | 1475 | | / |
| Theoretical assay | 237.3 | mg/g | / |

2. Results

2.1 Particle Size Distribution

It is presented in FIG. 3. The particle size distribution gives the following results:
D₁₀%: 88 μm
D₅₀%: 239 μm
D₉₀%: 435 μm.

2.2 Kinetics of Dissolution

They are studied under the same conditions as in example 1.

In addition to the milling, the presence of a surfactant accelerates the dissolution. The results are illustrated in FIG. 4.

After dissolving for 10 minutes, 26% of itraconazole is dissolved for the batch without surfactant, whereas 90% of itraconazole is dissolved for the batch with surfactant, for which virtually all the coprecipitated itraconazole is dissolved.

1. A process for the preparation of a particle composed of a coprecipitate applied as a layer around a neutral hydrophilic carrier by spraying an organic solution over said neutral hydrophilic carrier, said solution comprising at least one active substance, one surface-active agent and one hydrophilic polymer, wherein the spraying of the whole of the solution is carried out in at least two separate stages, each of these stages being followed systematically by a stage of milling the product obtained on conclusion of the preceding stage.

2. The process for the preparation of the particles as claimed in claim 1, wherein it comprises the following stages:
a) preparing an organic solution comprising the active substance, the hydrophilic polymer and the surface-active agent,
b) spraying a portion of the solution obtained in a) over the neutral hydrophilic carriers,
c) milling the particles obtained in stage b),
d) spraying the remaining amount of the organic solution over the particles milled in stage c)
e) final milling of the particles obtained in stage d).

3. The process for the preparation of the particles as claimed in claim 1, wherein the spraying/milling sequence (stages b to d) is repeated one or more times.

4. The process for the preparation of the particles as claimed in claim 1, wherein it additionally comprises a drying stage either after each spraying stage, before milling, or immediately after the milling.

5. The process for the preparation of the particles as claimed in claim 1, wherein the inert hydrophilic carrier is composed of any chemically and pharmaceutically inert excipient existing in the crystalline or amorphous particulate form and preferably chosen from the group consisting of sugar derivatives, celluloses and their mixtures.

6. The process for the preparation of the particles as claimed in claim 1, wherein the hydrophilic polymer is chosen from the group consisting of polyvinylpyrrolidones, in particular polymers with a molecular weight of between 10 000 and 50 000, cellulose derivatives, preferably hydroxypropylmethylcellulose, hydroxypropylcellulose, hydroxymethyl-cellulose, hydroxypropylmethylcellulose phthalate or hydroxypropylmethylcellulose acetate/succinate, acrylic polymers and polyethylene glycols.

7. The process for the preparation of the particles as claimed in claim 1, wherein the surface-active agent is chosen from the group consisting of cationic, anionic, nonionic and amphoteric agents, alone or as a mixture.

8. The process for the preparation of the particles as claimed in claim 1, wherein the organic solvent is chosen from the group consisting of ethanol, isopropanol, tetrahydrofuran, isopropyl ether, acetone, methyl ethyl ketone, methylene chloride and the mixtures of these solvents.

9. The process for the preparation of the particles as claimed in claim 1, wherein the spraying stages are carried out in a coating pan, in a perforated pan coater or in a fluidized bed.

10. A particle composed of a coprecipitate which is applied as a layer around a carrier and which comprises at least one active substance, one surface-active agent and one hydrophilic polymer, wherein it is capable of being obtained by spraying a solution comprising at least one active substance, one surface-active agent and one hydrophilic polymer, said spraying being carried out at least in two separate stages, said stages each being followed by a milling stage.

11. The particle as claimed in claim 10, wherein the active substance is present in the particle in a proportion which can vary between 1 and 60% by weight.

12. The particle as claimed in claim 10, wherein the inert hydrophilic carrier is present in a proportion which can range up to 95% by weight.

13. The particle as claimed in claim 10, wherein the hydrophilic polymer/active principle ratio by weight is between 10/1 and 1/2.

14. The particle as claimed in claim 10, wherein the surface-active agent is present in a proportion which can vary between 0. 1 and 20% by weight, with respect to the total weight obtained.

15. The particle as claimed in claim 10, wherein the unit particle size of the inert hydrophilic carrier can be between 50 and 500 μm.

16. The particle as claimed in claim 15, wherein the unit particle size of the inert hydrophilic carrier can be between 90 and 200 μm.

17. A pharmaceutical form, wherein it comprises at least one particle as claimed in claim 10, optionally in combination with pharmaceutically acceptable excipients.
